(19) United States
(12) Patent Application Publication
(10) Pub. No.: US 2025/0339665 A1
(43) Pub. Date: Nov. 6, 2025

(54) INTRA-THORACIC CARDIAC ASSIST DEVICES

(71) Applicant: EDWARDS LIFESCIENCES CORPORATION, Irvine, CA (US)

(72) Inventors: Sam Chitsaz, Walnut Creek, CA (US); Arnold Cruz Tuason, Claremont, CA (US); Emil Karapetian, Huntington Beach, CA (US)

(21) Appl. No.: 19/271,600

(22) Filed: Jul. 16, 2025

Related U.S. Application Data
(63) Continuation of application No. PCT/US2024/013234, filed on Jan. 26, 2024.
(60) Provisional application No. 63/482,172, filed on Jan. 30, 2023.

(51) Int. Cl. A61M 60/295; A61M 60/191; A61M 60/289; A61M 60/47; A61M 60/497; A61M 60/515

(52) U.S. Cl. CPC A61M 60/295; A61M 60/191; A61M 60/289; A61M 60/47; A61M 60/497; A61M 60/515

(57) ABSTRACT

A system for improving cardiac performance includes a delivery tube and an expandable implant sized for delivery within the delivery tube. The expandable implant is adjustable between an expanded form and a compressed form. In the expanded form, the expandable implant is sized and shaped to extend beyond the delivery tube for compressing a portion of a heart. The expandable implant may take the form of a balloon and may be inflated and deflated during use. A sensor is preferably provided for measuring cardiac rhythm, wherein the expandable implant moves in synchronization with the measured cardiac rhythm. The sensor is preferably coupled to the expandable implant. The delivery tube may be sized for advancement through a thoracic cavity.

### RELATED APPLICATION

This application is a continuation of International Patent Application No. PCT/US2024/013234, filed Jan. 26, 2024, which claims the benefit of U.S. Provisional Application No. 63/482,172. filed Jan. 30, 2023, the complete disclosures of which are hereby incorporated by reference in their entireties.

### BACKGROUND

The present disclosure generally relates to the field of medical procedures. Positive pressure ventilation used during surgery requiring general anesthesia can be detrimental to cardiac function and can generally have a particularly negative impact on the right ventricle. Patients with pulmonary hypertension and/or right heart disease can be especially susceptible to this phenomenon, contributing to high mortality rates during cardiac and non-cardiac surgery requiring general anesthesia.

### SUMMARY

Described herein are one or more methods and/or devices to assist cardiac function.

Some implementations of the present disclosure relate to a method of assisting cardiac performance including: accessing a thoracic cavity near a heart with an access tube; delivering, via the access tube, an expandable implant in a compressed form into the thoracic cavity; and expanding the expandable implant to cause compression of the heart.

In some aspects, the techniques described herein relate to a method, further including monitoring cardiac rhythm using one or more sensors attached to the access tube or expandable implant.

In some aspects, the techniques described herein relate to a method, further including expanding the expandable implant in synchronization with the cardiac rhythm.

In some aspects, the techniques described herein relate to a method, wherein at least one pressure sensor is situated at an exterior surface of the access tube.

In some aspects, the techniques described herein relate to a method, further including accessing the thoracic cavity via an anterior mediastinum with the access tube.

In some aspects, the techniques described herein relate to a method, further including positioning a distal end of the access tube at or near a right ventricle of the heart within the thoracic cavity.

In some aspects, the techniques described herein relate to a method, wherein expanding the expandable implant includes delivering a fluid or gas into the expandable implant via the access tube.

In some aspects, the techniques described herein relate to a method, wherein the expandable implant includes a balloon.

In some aspects, the techniques described herein relate to a method, wherein the expandable implant includes a coil disposed inside the balloon.

In some aspects, the techniques described herein relate to a method, wherein the expandable implant includes a mesh tube disposed inside the balloon.

In some aspects, the techniques described herein relate to a method, wherein the expandable implant includes a ridged device configured to expand longitudinally and not laterally.

In some aspects, the techniques described herein relate to a method, wherein the expandable implant includes a scissor jack mechanism.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loudspeakers, headphones, pressure transducers, temperature transducers, or using any combination of suitable technologies.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates a vertical/frontal cross-sectional view of an example thoracic cavity having various features/anatomy relevant to certain aspects of the present inventive disclosure.

FIG. 2 provides an overhead view of various anatomy of a thoracic cavity and illustrates an example method for managing intra-thoracic pressures in accordance with one or more examples.

FIG. 3 illustrates an example pressure management system for selectively driving pressure within a thoracic cavity, in accordance with one or more examples.

FIG. 4 illustrates an example pressure management process involving delivering one or more access tubes to a thoracic cavity and/or at or near a heart of a patient in accordance with one or more examples.

FIG. 5 illustrates example pressure regulation using an expanded and/or inflated cardiac assist device delivered via an access tube into and/or within the thoracic cavity in accordance with one or more examples.

FIG. 6 illustrates an example assist device and/or actuator configured for delivery via an access tube (e.g., catheter and/or delivery shaft) in accordance with one or more examples.

FIG. 7 illustrates an example assist device and/or actuator configured for delivery via an access tube (e.g., catheter and/or delivery shaft) in accordance with one or more examples.

FIG. 8 illustrates an example assist device and/or actuator configured for delivery via an access tube (e.g., catheter and/or delivery shaft) in accordance with one or more examples.

FIG. 9 illustrates an example assist device and/or actuator configured for delivery via an access tube (e.g., catheter and/or delivery shaft) in accordance with one or more examples.

FIG. 10 illustrates an example assist device and/or actuator configured for delivery via an access tube (e.g., catheter and/or delivery shaft) in accordance with one or more examples.

FIG. 11 provides a flowchart illustrating an example process for assisting cardiac performance in accordance with one or more examples.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Certain standard anatomical terms of location are used herein to refer to certain device components/features and to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” “under,” “over,” “topside,” “underside,” and similar terms, are used herein to describe a spatial relationship of one device/element or anatomical structure to another device/element or anatomical structure, it is understood that these terms are used herein for ease of description to describe the positional relationship between clement(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

The present disclosure relates to systems, devices, and methods for assisting cardiac performance within patients. In some cases, the systems, devices, and/or methods described herein can be used to augment cardiac output in surgical, intensive care unit, chronic, and/or emergency settings. Some examples may be of particular benefit to patients experiencing pulmonary hypertension and/or other cardiac conditions. For example, patients experience pulmonary hypertension can require mechanical pressure ventilation, which can involve pushing air into the patients' lungs. In such cases, the lungs may expand while the thoracic cavity does not, which is in contrast to normal breathing, in which thoracic cavity expansion causes corresponding lung expansion. The expansion of the lungs during mechanical pressure ventilation can cause increased pressure in the thoracic cavity, which can negatively impact cardiac function.

Some examples provide methods for driving thoracic pressure and/or ventilating thoracic pressure based on measured pressure values. Pressure within the thoracic cavity can be sensed to determine whether pressure should be increased or decreased to assist with cardiac function.

Some examples of the present disclosure may involve gaining access to the thoracic cavity using an access tube (e.g., a chest tube). Pressure pulsation may be applied to one or more cardiac assist devices and/or implants through the access tube to directly compress the heart without needing to compress the thoracic cavity through the rib cage. In some cases, compression of the thoracic cavity through the rib cage can cause trauma.

In some examples, a cardiac rhythm monitor may be used to synchronize pressure pulsation with a patient's cardiac function. In some examples, pressure pulsation and/or cardiac monitoring may be used for patient with pulmonary hypertension and/or undergoing surgery.

### Thoracic Physiology

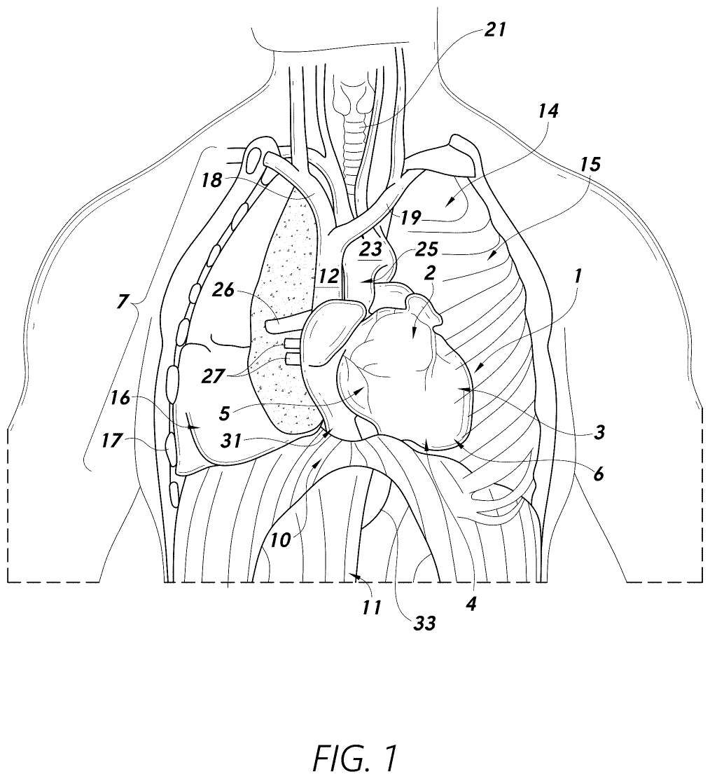

The anatomy of the thoracic cavity and surround anatomy is described below to assist in the understanding of certain inventive concepts disclosed herein. FIG. 1 illustrates a vertical/frontal cross-sectional view of an example thoracic cavity 7 having various features/anatomy relevant to certain aspects of the present inventive disclosure. The thoracic cavity 7 (i.e., chest cavity) is an area of anatomy enclosed by the ribs 15, 17, the vertebral column, and the sternum 42 (i.e., breastbone). The thoracic cavity 7 is separated from the abdominal cavity by the diaphragm 10, which is a respiration muscle that contracts rhythmically and continually. This contraction creates a vacuum within the thoracic cavity 7, which pulls air into the lungs 14, 16. The right lung 16 is located on the right side of the thoracic cavity 7 and is shorter than the left lung 14, which is located on the left side of the thoracic cavity 7.

The thoracic cavity 7 also includes the heart 1, the vessels transporting blood between the heart 1 and the lungs, the great arteries bringing blood from the heart 1 out into general circulation, and the major veins into which the blood is collected for transport back to the heart 1. The right brachiocephalic vein 18 and left brachiocephalic vein 19 convey blood from the head and neck, upper limbs, and thorax to the heart 1 and unite at the level of the inferior border of the 1^{st} right costal cartilage to form the superior vena cava 12. The inferior vena cava 31 drains venous blood from the lower trunk, abdomen, pelvis, and lower limbs to the right atrium 5 of the heart 1.

The heart 1 is covered by a fibrous membrane sac called the pericardium that blends with the trunks of the vessels running to and from the heart 1. The thoracic cavity 7 also contains the esophagus 11, the channel through which food is passed from the throat to the stomach.

The thoracic cavity 7 is lined with a serous membrane, referred to as the parietal pleura, which exudes a thin fluid. The membrane continues over the lung, where it is called the visceral pleura, and over part of the esophagus 11, the heart 1, and the great vessels, as the mediastinal pleura. Because the atmospheric pressure between the parietal pleura and the visceral pleura is less than that of the outer atmosphere, the two surfaces tend to touch.

The heart 1 includes four chambers, namely the left atrium 2, the left ventricle 3, the right ventricle 4, and the right atrium 5. The heart 1 sits atop the diaphragm 10 and the apex 6 of the heart 1 is close to the anterior surface of the thoracic cavity 7. In terms of blood flow, blood generally flows from the right ventricle 4 into the pulmonary artery 26 via the pulmonary valve, which separates the right ventricle 4 from the pulmonary artery 26 and is configured to open during systole so that blood may be pumped toward the lungs 14, 16 and close during diastole to prevent blood from leaking back into the heart 1 from the pulmonary artery 26. The pulmonary artery 26 carries deoxygenated blood from the right side of the heart 1 to the lungs 14, 16. Pulmonary veins 27 deliver oxygenated blood from the lungs 14, 16 to the left atrium 2 of the heart 1.

The heart valves may generally comprise a relatively dense fibrous ring, referred to as the annulus, as well as a plurality of leaflets or cusps attached to the annulus. Generally, the size of the leaflets/cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Dysfunction of a heart valve and/or associated leaflets (e.g., pulmonary valve dysfunction) can result in valve leakage and/or other health complications.

During normal operation, the thoracic cavity 7 increases in size when a breath is drawn. The increase of the thoracic cavity 7 creates a vacuum within the thoracic cavity 7 which draws air into the lungs 14, 16, expanding the lungs 14, 16. The trachea 21 is a large membranous tube extending from the larynx to the bronchial tubes and conveying air to and from the lungs 14, 16. When a patient is treated with intubated and/or mechanical ventilation, air may be pushed into the lungs. The right side of the heart (e.g., the right ventricle 4) generally has a lower pressure than the left side of the heart (e.g., the left ventricle 3). As a result, the right ventricle 4 can have difficulty in cases of increased pressure. For example, vessels in the pulmonary vasculature can become compacted. When the right ventricle 4 is surrounded by increased pressure, it can have difficulty filling with blood, which can in turn compromise return blood from the right ventricle 4.

The aortic arch 23 is a section of the aorta 46 between the ascending aorta 25 and the descending aorta 33. The aorta 46 helps distribute blood from the heart 1 to the head and upper extremities. The azygos vein 45 is a unilateral vessel that ascends up the thoracic vertebral column, carrying deoxygenated blood from the posterior chest and abdominal walls. The esophagus 47 is part of the alimentary canal that connects the throat to the stomach.

Health Conditions and Treatments Associated with Intra-Thoracic Pressure

Mechanical ventilation of the thoracic cavity 7 is a treatment method sometimes used during surgery requiring general anesthesia. Mechanical ventilation can involve increasing thoracic pressure during inspiration. In some types of mechanical ventilation, expiration may be ended when an ambient pressure is reached within the thoracic cavity 7. Positive pressure ventilation is a type of mechanical ventilation in which a positive pressure (e.g., higher than ambient pressure) is maintained even at the end of expiration. Mechanical ventilation can increase afterload and/or reduce preload on the right ventricle, with positive pressure ventilation generally having a relatively larger effect than other types of mechanical ventilation.

In some cases, mechanical ventilation can be detrimental to cardiac function. For example, the positive pressure can cause elevated intra-thoracic pressures that can be detrimental to cardiac function, generally with a larger impact on the right ventricle. These effects can be particularly harmful for patients suffering from pulmonary hypertension. Pulmonary hypertension is a disease characterized by high blood pressure that affects the arteries in the lungs and on the right side of the heart 1 (e.g., the right lung 16 and/or pulmonary artery 26). In some cases of pulmonary hypertension, blood vessels in the lungs 14, 16 are narrowed, blocked, or destroyed. Patients with pulmonary hypertension and/or right heart disease can be especially susceptible to damage to cardiac function from elevated intra-thoracic pressures, which can contribute to high mortality rates during cardiac and/or non-cardiac surgery requiring general anesthesia.

The challenges associated with positive pressure ventilation can include the following. Positive pressure ventilation can increase pressure within the thoracic cavity 7. This pressure can cause compression of the right ventricle 4 and/or other areas of the heart 1 and/or can prevent the right ventricle 4 from filling properly (e.g., reducing right ventricle 4 preload). The increased intra-thoracic pressure can also collapse the pulmonary vasculature, causing elevated resistance that can lead to an increase in the right ventricle 4 afterload. Because the right ventricle 4 is highly sensitive to increases in afterload, this can have detrimental effects on right ventricle 4 function, especially in patients with pulmonary hypertension and/or who may have compromised right ventricle 4 function and/or right heart failure.

During normal conditions, expansion of the ribs 15, 17 causes expansion of the lungs 14, 16. In various surgical and/or non-surgical ventilation procedures, patients may be unconscious and/or otherwise unable to expand their ribs 15, 17. Accordingly, ventilation of the lungs 14, 16 may not involve corresponding expansion of the thoracic cavity 7. Some treatments may involve squeezing the heart 1 to assist the heart 1 in pumping. However, the heart 1 may be required to pump through narrow vasculature in such cases and may be required to work harder than under normal conditions.

Cardiac arrest, cardiogenic shock, and acute decompensated heart failure are medical emergencies that can require immediate treatment as the heart is unable to pump enough blood to maintain vital bodily functions. Mechanical cardiac support can be an important part of the treatment. With the exception of chest compressions for patients under cardiac arrest, cardiac support treatments can require (e.g., at a Cath lab) enabling physicians to use mechanical support devices such as the Impella device. A less invasive approach to mechanically support cardiac function for patients with acute decompensated heart failure, cardiogenic shock, and/or cardiac arrest can substantially improve outcomes.

### Managing Intra-Thoracic Pressure

Examples described herein can advantageously prevent or reduce the detrimental effects of increased intra-thoracic pressure by actively maintaining favorable intra-thoracic pressures. Some devices and/or methods described herein can also mechanically assist cardiac function using synchronized pressure pulsation.

The various methods and/or devices described herein for managing intra-thoracic pressures can have various advantages. For example, breath and/or ventilation rate can be monitored and incorporated into a thoracic pressure regulation algorithm. Multiple access points can be used for improved pressure regulation. Some examples can advantageously augment cardiac output in surgical, intensive care unit, chronic, and/or emergency settings. Gas (e.g., air, CO₂, nitrogen, etc.) can be heated and/or humidified to avoid adhesions and tissue drying, similar to laparoscopic insufflation. Hemodynamic pressures can be monitored and used to optimize pressure regulation for cardiac function. For example, hemodynamic pressure, heart rate, and/or ventilation rate can be used as feedback to adjust driving pressures for optimizing ventricular support.

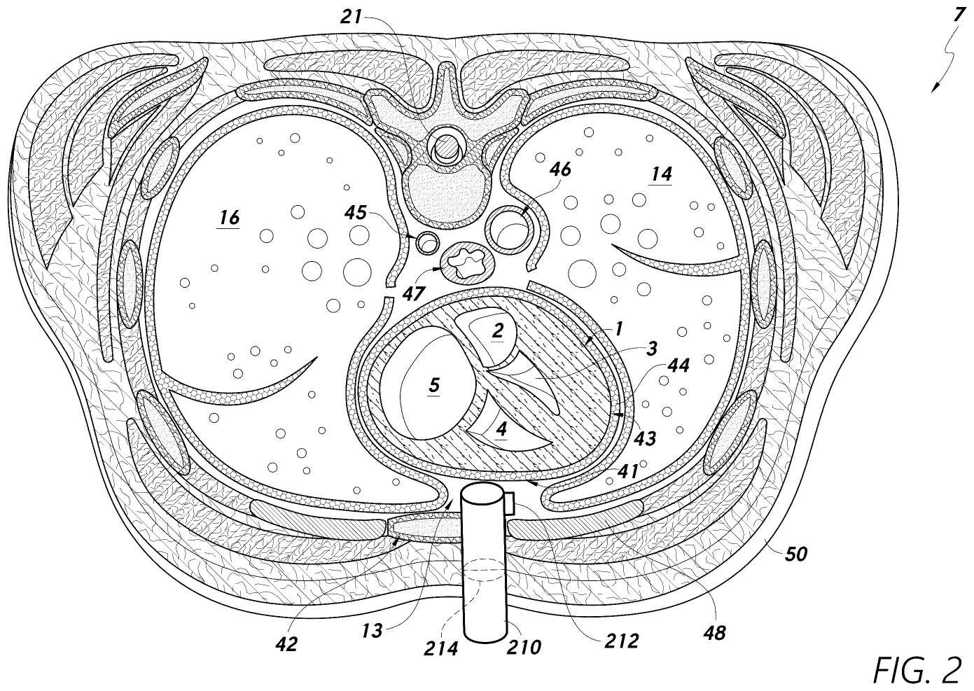

FIG. 2 provides an overhead view of various anatomy of a thoracic cavity 7 and illustrates an example method for managing intra-thoracic pressures in accordance with one or more examples. As shown in Figure s, a conical sac of fibrous tissue known as the pericardial sac or pericardium 41 surrounds the heart 1 and the roots of the great blood vessels. A serous membrane known as the epicardium 43 forms an innermost layer of the pericardium 41 and an outer surface of the heart 1. A pericardial cavity 44 is enclosed by the pericardium 41 and contains the heart 1.

The thoracic wall 50 consists of a bony framework that is held together by twelve thoracic vertebrae posteriorly which give rise to ribs that encircle the lateral and anterior thoracic cavity 7. The ribs connect to the sternum 42 with cartilage. The sternum 42 is a flat bone that sits at the front of the chest and/or thoracic cavity 7 and is part of the rib cage. The costal cartilage (including the 5^{th} costal cartilage 48) are bars of hyaline cartilage which serve to prolong the ribs forward and contribute to the elasticity of the walls of the thorax.

As illustrated in FIG. 2, some example methods of regulating pressure within the thoracic cavity 7 can involve using an access tube 210 to access the thoracic cavity 7. While a chest tube access method (e.g., commonly used to treat pneumothorax) is shown in FIG. 2, the thoracic cavity 7 may be accessed by any suitable access point. In some examples, the access tube 210 can be placed into the thoracic cavity 7 via the anterior mediastinum 13, which is adjacent to the right ventricle 4 and can optimize systolic compression assist. The access tube 210 can be inserted through the skin below and/or adjacent to the sternum 42 and/or the 5^{th} costal cartilage 48.

In some examples, the access tube 210 may comprise and/or may be configured to deliver one or more pressure sensors 212 for use in sensing pressure within the thoracic cavity 7. For example, one or more sensors 212 may be situated along an exterior and/or interior (e.g., lumen-facing) surface of the access tube 210. In some examples, one or more pressure sensors 212 may be outside the thoracic cavity 7 and/or otherwise positioned along the access tube 210. The position of the access tube 210 at least partially within the thoracic cavity 7 may allow the one or more pressure sensors 212 to detect thoracic pressure from any position along the access tube 210.

The access tube 210 and/or other delivery systems may be used to deliver one or more valves 214 configured to control pressure input and/or output to/from the thoracic cavity 7. For example, a valve 214 may be situated at least partially within a lumen of the access tube 210. The valve 214 may be configured to release pressure from the thoracic cavity 7 in response to the pressure sensor 212 detecting elevated intra-thoracic pressure (e.g., in response to pressure readings exceeding a threshold and/or control value). Thus, the valve 214 may be configured to prevent preload reduction and/or afterload increase to the right ventricle 4 and/or to prevent decompensation.

In some examples, pressure regulation may be performed without use of sensors 212. For example, a system may be configured to passively regulate thoracic pressure using one or more pre-set pressure check valves 214. The valve 214 may be configured to naturally release in response to pressure (e.g., on the thoracic side of the valve 214) exceeding a pre-set pressure value for the valve 214. In some examples, the pre-set pressure value may be approximately equal to an ambient pressure value.

The valve 214 may be a pressure release valve configured to release gas buildup from around the lungs. Gas released through the valve 214 may be released out of the body (e.g., through the access tube 210. The valve 214 may comprise a ball valve, disc valve, electrically controlled valve, and/or spring-biased valve.

In some examples (e.g., for patients experiencing acute decompensated heart failure or cardiogenic shock), a cardiac rhythm monitor may be used to allow for synchronization of pressure inputs with cardiac function. For example, pressure pulses may be applied via the access tube 210 in synchronization with a patient's heartbeat and/or cardiac rhythm. In some examples, once access to the thoracic cavity 7 and/or cardiac rhythm is established, a lower (or possibly negative) pressure may be applied to aid ventricular filling and/or a higher pressure may be applied to aid ventricular contraction.

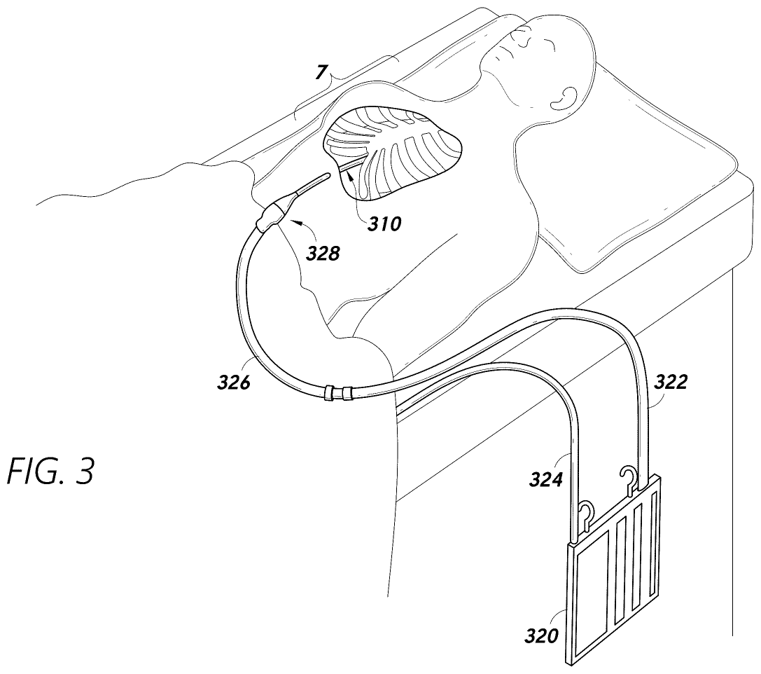

FIG. 3 illustrates an example pressure management system for selectively driving pressure within a thoracic cavity 7, in accordance with one or more examples. The system may comprise a control module 320 comprising a compressor pump and/or a vacuum pump and/or configured to control a compressor pump and/or vacuum pump. The control module 320 may comprise one or more inputs configured to receive a pressure drive tube 322 and/or a cardiac synchronization input 324. The cardiac synchronization input 324 may comprise a tube connected to a finger cuff and/or other device configured to monitor and/or measure cardiac rhythm and/or pressure features. For example, the cardiac synchronization input 324 may be configured to detect the patient's heartbeat and/or breath rate. In some examples, the patient's breath and/or ventilation rate can be monitored and/or incorporated into a thoracic pressure regulation algorithm and/or determination process.

The pressure drive tube 322 may connect to and/or extend into a delivery tube 326 configured to attach to and/or extend into an access tube 310. In some examples, the access tube 310, delivery tube 326, and/or pressure drive tube 322 may comprise a single catheter and/or tube. The delivery tube 326 may be configured to convey pressure from the control module 320 to the access tube 310 and/or convey pressure feedback from the access tube 310 to the control module 320. A connector 328 may be used to couple the access tube 310 to the delivery tube 326.

In some examples, the access tube 310 may be configured to establish a sealed entry though the patient's skin and/or into the thoracic cavity 7. While only a single access point and/or access tube 310 is shown in FIG. 3, multiple access points and/or multiple access tubes 310 may be used to regulate pressure (e.g., simultaneously) throughout the thoracic cavity 7. In some examples, the access tube(s) 310 may comprise one or more sensors configured to sense pressure within the thoracic cavity 7.

The pressure drive tube 322, delivery tube 326, and/or access tube 310 may be configured to convey gas (e.g., air, CO₂, nitrogen, etc.) into the thoracic cavity 7 and/or to convey gas out of the thoracic cavity 7. In some examples, gas may be heated and/or humidified prior to delivery into the thoracic cavity 7 to avoid adhesions and/or tissue drying (e.g., similar to laparoscopic insufflation). Hemodynamic pressures can be monitored and/or used to optimize pressure regulation for cardiac function.

In some examples, pressure input (e.g., pressure pulses from the control module 320 may be synchronized with the patient's natural cardiac rhythm (e.g., heartbeat and/or breath rate) using data received via the cardiac synchronization input 324 tube via a finger cuff, patch, and/or other cardiac rhythm sensor. For example, the control module 320 may be configured to convey pressure pulses via the pressure drive tube 322 at a first rhythmic event (e.g., a breath in) and/or to pull and/or ventilate gas from the thoracic cavity at a second rhythmic event (e.g., a breath out). In some examples, the control module 320 may not actively suction gas out of the thoracic cavity 7 and/or the access tube 310 may provide ventilation to allow natural release of gas from the thoracic cavity 7 when pressure within the thoracic cavity 7 is high. Actions performed by the control module 320 may not necessarily be performed at each rhythmic event. For example, the control module 320 may not drive pressure pulses with each heartbeat and/or breath but may drive pressure pulses in a periodic manner (e.g., every third heartbeat and/or breath).

Some implementations of the present disclosure relate to methods of assisting right ventricle performance and/or various devices for performing such methods. Some methods may involve accessing the thoracic cavity near the right ventricle of the heart with an access tube carrying an expandable actuator. Some examples involve causing compression of the right ventricle periodically and/or during systole using the actuator. The actuator can comprise any of a variety of devices described herein, which can include a balloon actuator, a bellows-type and/or ridged device, a covered coil/spring, a covered braided wire form, and/or a scissor jack and/or piston.

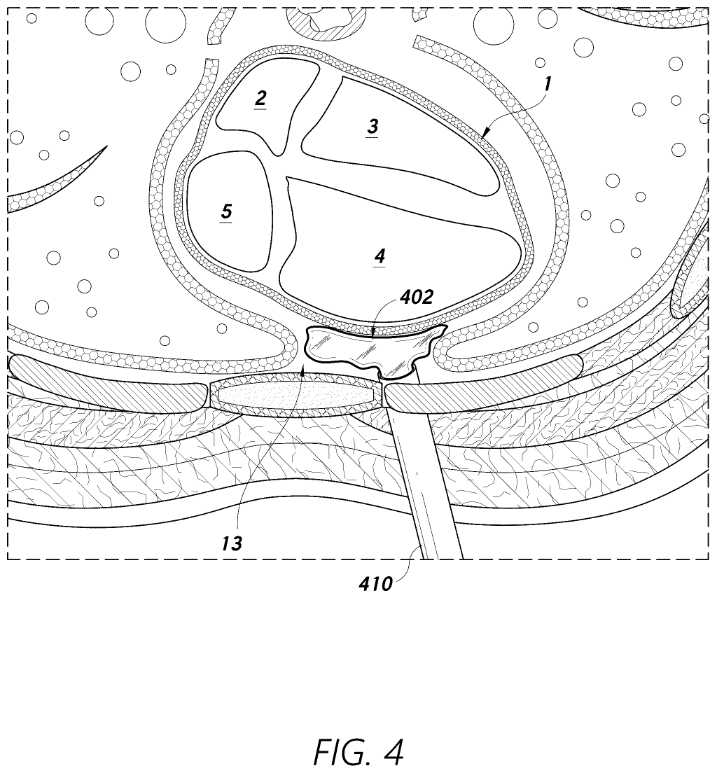

FIG. 4 illustrates an example pressure management process involving delivering one or more access tubes 410 to a thoracic cavity and/or at or near a heart 1 of a patient in accordance with one or more examples. As shown in FIG. 4, an access tube 410 may gain access to a thoracic cavity via any suitable means. For example, the access tube 410 may be configured to be passed through a cavity 13 and/or opening into the thoracic cavity (e.g., the anterior mediastinum) to simplify a delivery process. The anterior mediastinum 13 may contain no major structure and/or may accommodate loose connective tissue. The anterior mediastinum 13 may advantageously provide access at or near the right ventricle 4 of the heart 1. In some examples, the access tube 410 may be delivered between, above, and/or below one or more ribs. For example, the access tube 410 may be configured for delivery below the 5^{th} costal cartilage. The access tube 410 may be delivered adjacent to and/or to the left or right of the sternum.

While the access tube 410 is shown being delivered through the anterior mediastinum 13, the access tube 410 may be configured for delivery at any suitable location. Moreover, while the access tube 410 is shown being delivered proximate to the heart 1, the access tube 410 may be configured for delivery to any portion of the thoracic cavity. In some examples, the access tube 410 and/or a distal end of the access tube 410 (e.g., an open end) may be positioned at or near the right ventricle 4 of the heart 1 to affect pressure and/or volume of the right ventricle 4 and/or the heart 1.

In some examples, the access tube 410 may be configured for delivery of one or more cardiac assist devices 402. Cardiac assist device 402 can have any of a variety of forms and/or may be at least partially expandable. FIG. 4 illustrates the device 402 in an at least partially compressed and/or deflated form. For example, the device 402 can comprise a generally flexible sheet of material (e.g., rubber, fabric, etc.) forming an at least partially closed circle and/or loop. The device 402 may be configured to be filled with one or more gases and/or fluids. Compression and/or deflation of the device 402 may facilitate delivery and/or navigation of the device 402 through the access tube 410 and/or through the anterior mediastinum 13.

The device 402 may be extended at least partially beyond a distal end of the access tube 410 during and/or following delivery of the access tube 410 to a desired position within the body and/or thoracic cavity. In some examples, the device 402 may be pressed into contact with the heart 1 and/or other anatomy prior to inflation and/or expansion of the device 402. For example, the device 402 may be placed into contact with the heart 1 at or near a right ventricle 4 portion of the heart 1. The device 402 may be at least partially flexible and/or may be configured to conform around a curvature of the heart 1 in the deflated and/or compressed form.

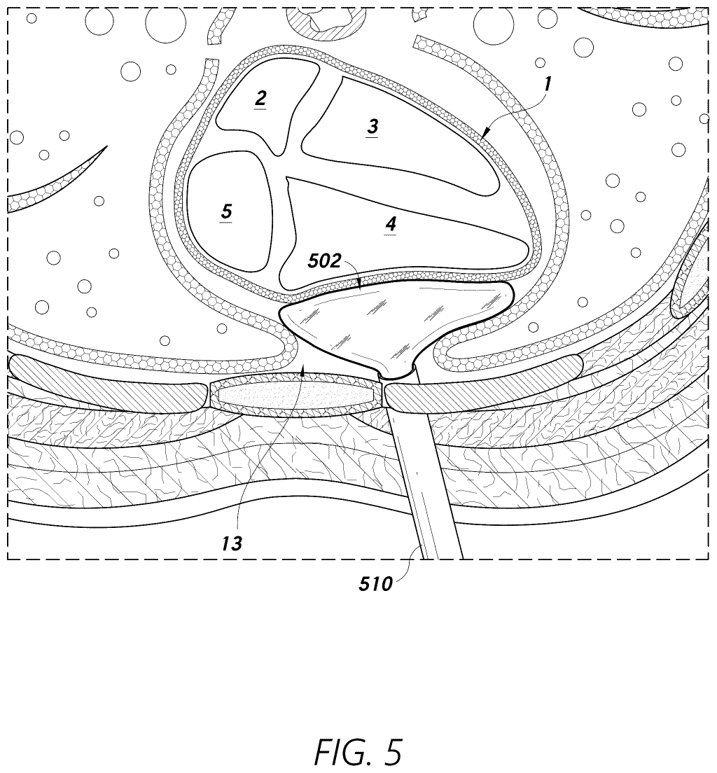

FIG. 5 illustrates example pressure regulation using an expanded and/or inflated cardiac assist device 502 delivered via an access tube 510 into and/or within the thoracic cavity in accordance with one or more examples. The access tube 510 may be configured to direct the device 502 towards the heart 1.

Cardiac assist device 502 can have any of a variety of forms and/or may be at least partially expandable. FIG. 5 illustrates the device 502 in an at least partially expanded and/or inflated form. For example, the device 502 can comprise a generally flexible sheet of material (e.g., rubber, fabric, etc.) forming an at least partially closed circle and/or loop. The device 502 may be configured to be filled with one or more gases and/or fluids to cause expansion of the device 502. Expansion and/or inflation of the device 502 may cause pressure and/or shaping at the heart 1 and/or right ventricle 4.

The device 502 may be extended at least partially beyond a distal end of the access tube 510 during and/or following delivery of the access tube 510 to a desired position within the body and/or thoracic cavity. In some examples, the device 502 may be pressed into contact with the heart 1 and/or other anatomy prior to inflation and/or expansion of the device 502. For example, the device 502 may be placed into contact with the heart 1 at or near a right ventricle 5 portion of the heart 1. The device 502 may be at least partially flexible and/or may be configured to conform around a curvature of the heart 1 in the deflated and/or compressed form. For example, the device 502 may be delivered in an at least partially deflated state and/or form into the thoracic cavity. The device 502 may be configured to be selectively expanded and/or inflated. For example, the device 502 may be configured to inflated in synchronization with a cardiac rhythm and/or cycle.

In some examples, a cardiac rhythm monitor may be used to synchronize pressure pulsation and/or inflation of the device 502 with a patient's cardiac function. Once access and/or cardiac rhythm is/are established, the device 502 may be selectively inflated and/or deflated in rhythm with the cardiac rhythm. For example, the device 502 may be expanded and/or inflated during systole and/or compressed and/or deflated during diastole.

In some examples, expansion and/or inflation of the device 502 via the access tube 510 may be performed using one or more valves and/or sensors. For example, the access tube may comprise one or more valves (e.g., situated at least partially within a lumen of the access tube 510) configured to open to allow delivery of gas and/or fluid in response to pressure within the thoracic cavity falling below a control value and/or in synchronization with cardiac rhythm. For example, the control valve may be a check valve configured to naturally open in response to pressure beyond a distal end of the access tube 510 (e.g., within the thoracic cavity) falling below an ambient pressure and/or other control pressure value. In some examples, one or more sensors (e.g., situated at least partially within a lumen and/or along an exterior of the access tube 510) may be configured to sense pressure within the thoracic cavity. In some examples, a first pressure sensor may be configured to detect pressure within the thoracic cavity and/or a second pressure sensor (e.g., situated outside the body) may be configured to detect pressure outside the body (e.g., an ambient pressure) and/or the sensed pressures and/or a pressure drop between the two sensors may be used to activate the one or more valves.

The device 502 may have any of a variety of forms and/or may comprise one or more actuators configured to mechanically exert force on the heart 1 and/or right ventricle 4. In the example shown in FIG. 5, the device 502 comprises an inflatable balloon and/or similar actuator. The device 502 may be configured to inflate to a generally spherical shape. However, resistive force of the heart 1 and/or surrounding anatomy in the thoracic cavity may prevent the device 502 from expanding to a fully spherical form. Accordingly, the device 502 may be configured to expand to a partial spherical form. In some examples, an actuator may be configured to cause compression of the right ventricle 4 periodically and/or during systole.

FIG. 6 illustrates an example assist device 602 and/or actuator configured for delivery via an access tube 610 (e.g., catheter and/or delivery shaft) in accordance with one or more examples. The device 602 can comprise an inflatable and/or expandable balloon. In some examples, the device 602 may be configured for inflation of one or more gases and/or fluids delivered via the tube 610. The device 602 may be configured to inflate to a generally spherical shape. However, the device 602 can have a generally flexible and/or pliable form and/or may be configured to conform to various environments and/or anatomies. The device 602 can be composed of various suitable materials, which can include compliant and/or non-compliant materials. Various materials of the device 602 may be configured to stretch and/or expand in response to filling of gases and/or fluids. In some examples, the device 602 (e.g., an inflatable and/or expandable balloon) may be incorporated with other devices and/or structures as described herein. For example, the device 602 may be at least partially enclosed by a generally rigid structure configured to prevent and/or mitigate lateral expansion of the device 602 and/or configured to allow and/or facilitate longitudinal expansions of the device 602.

In some examples, the device 602 and/or tube 610 can comprise one or more additional features, which can include one or more pressure sensors configured to sense changes in preload and/or afterload, radiopaque markers, electrical leads for ventricular pacing, defibrillation, and/or sensing cardiac rhythm, and/or permanent and/or temporary tissue anchors configured to facilitate placement and/or positioning of the device 602.

The device 602 may be configured to be selectively expanded and/or inflated. For example, the device 602 may be configured to inflated in synchronization with a cardiac rhythm and/or cycle. For example, the one or more sensors may be configured to generate and/or transmit data relating to cardiac rhythm (e.g., systole and/or diastole) and/or the device 602 may be automatically expanded and/or compressed on a periodic basis in synchronization with the cardiac rhythm.

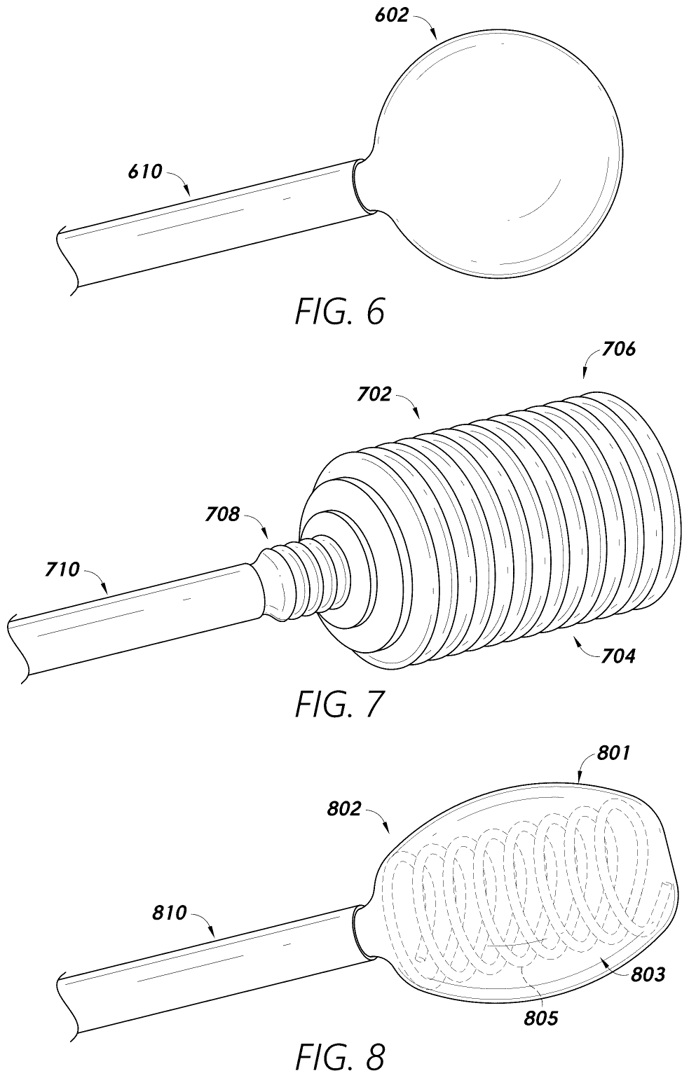

FIG. 7 illustrates an example assist device 702 and/or actuator configured for delivery via an access tube 710 (e.g., catheter and/or delivery shaft) in accordance with one or more examples. The device 702 can comprise an inflatable and/or expandable bellows-like mechanism. In some examples, the device 702 may be configured for inflation of one or more gases and/or fluids delivered via the tube 710. The device 702 may be configured to inflate to a generally elongate and/or tubular shape. The device 702 can have a generally rigid structure and/or may be configured to resist lateral expansion and/or to substantially expand longitudinally (e.g., along a direction of insertion from the tube 710). The device 702 can be composed of various suitable materials, which can include compliant and/or non-compliant materials. Various materials of the device 702 may be configured to stretch and/or expand in response to filling of gases and/or fluids.

The device 702 may be configured to direct injection of gas and/or fluid towards the heart 1 and/or right ventricle 4. In some examples, the device 702 may be longitudinally expandable and/or may not be laterally expandable. The device 702 may comprise ridges 704 at an exterior surface of the device 702 configured to facilitate expansion and/or compression of the device 702 in response to corresponding inflation and/or deflation of the device 702. The device 702 may comprise a distal portion 706 and/or a proximal portion 708. The distal portion 706 may have a greater diameter than the proximal portion 708. The distal portion 706 and/or proximal portion 708 may have a generally expandable structure and/or tubular form. In some examples, the distal portion 706 may have a ridged form (e.g., comprising ridged edges) and/or may be configured to expand longitudinally (e.g., away from the proximal portion 708) and/or may be configured to not expand laterally (e.g., perpendicularly to the proximal portion 708).

In some examples, the device 702 and/or tube 710 can comprise one or more additional features, which can include one or more pressure sensors configured to sense changes in preload and/or afterload, radiopaque markers, electrical leads for ventricular pacing, defibrillation, and/or permanent and/or temporary tissue anchors configured to facilitate placement and/or positioning of the device 702.

The device 702 may be configured to be selectively expanded and/or inflated. For example, the device 702 may be configured to inflated in synchronization with a cardiac rhythm and/or cycle. For example, the one or more sensors may be configured to generate and/or transmit data relating to cardiac rhythm (e.g., systole and/or diastole) and/or the device 702 may be automatically expanded and/or compressed on a periodic basis in synchronization with the cardiac rhythm.

FIG. 8 illustrates an example assist device 802 and/or actuator configured for delivery via an access tube 810 (e.g., catheter and/or delivery shaft) in accordance with one or more examples. The device 802 can comprise an inflatable and/or expandable balloon 801 and/or coil 803 (e.g., spring). In some examples, the device 802 may be configured for inflation of one or more gases and/or fluids delivered via the tube 810. The coil 803 may be configured to facilitate longitudinal expansion of the balloon 801. For example, the coil 803 may have a generally tubular form and/or may be configured to compress and/or expand longitudinally (e.g., along a direction of insertion and/or delivery of the device 802. The coil 803 may comprise a wire composed of one or more metals (e.g., Nitinol and/or other shape memory alloys) and/or other suitable materials. The coil 803 may comprise a number of windings 805 forming a helical and/or tubular shape. In some examples, the coil 803 may be configured to be compressed to a compact form in which the windings contact each other. The coil 803 may be configured to expand and/or to be expanded to the expanded form shown in FIG. 8, in which the windings 805 are not in contact with each other. As the coil 803 expands, the coil 803 may exert longitudinal force on the balloon 801 to cause the balloon 801 to form a generally oval shape, in which the balloon 801 has a greater length (e.g., longitudinal axis) than width (e.g., lateral axis). Thus, the coil 803 may be configured to direct expansion of the balloon 801 longitudinally.

The device 802 can comprise an inflatable and/or expandable balloon 801. In some examples, the balloon 801 may be configured for inflation of one or more gases and/or fluids delivered via the tube 810. The balloon 801 may be configured to inflate to a generally spherical shape. However, the balloon 801 can have a generally flexible and/or pliable form and/or may be configured to conform to various environments and/or anatomies. The balloon 801 can be composed of various suitable materials, which can include compliant and/or non-compliant materials. Various materials of the balloon 801 may be configured to stretch and/or expand in response to filling of gases and/or fluids. In some examples, the balloon 801 may have sufficient elasticity to apply compressive force to the coil 803. However, as the balloon 801 is inflated, the coil 803 may be allowed to expand and/or shape the balloon 801 into an elongate oval shape.

The coil 803 may be disposed at least partially and/or fully within the balloon 801. Accordingly, expansion and/or compression of the coil 803 may be configured to cause corresponding expansion and/or compression of the balloon 801. In some examples, the coil 803 (e.g., spring) may be configured to operate passively and/or in response to expansion and/or compression of the balloon 801. Deflation of the balloon 801 may be configured to create a vacuum within the balloon 801 and/or to cause compression of the coil 803.

In some examples, the device 802 and/or tube 810 can comprise one or more additional features, which can include one or more pressure sensors configured to sense changes in preload and/or afterload, radiopaque markers, electrical leads for ventricular pacing, defibrillation, and/or permanent and/or temporary tissue anchors configured to facilitate placement and/or positioning of the device 802.

The device 802 may be configured to be selectively expanded and/or inflated. For example, the device 802 may be configured to inflated in synchronization with a cardiac rhythm and/or cycle. For example, the one or more sensors may be configured to generate and/or transmit data relating to cardiac rhythm (e.g., systole and/or diastole) and/or the device 802 may be automatically expanded and/or compressed on a periodic basis in synchronization with the cardiac rhythm.

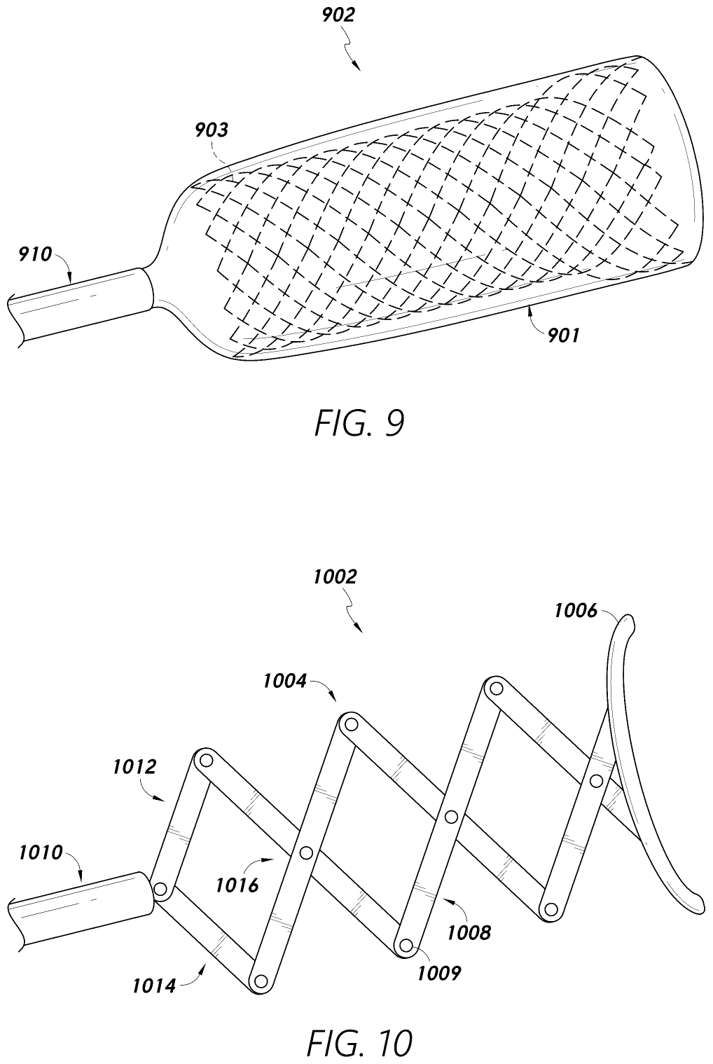

FIG. 9 illustrates an example assist device 902 and/or actuator configured for delivery via an access tube 910 (e.g., catheter and/or delivery shaft) in accordance with one or more examples. The device 902 can comprise an inflatable and/or expandable balloon 901 and/or mesh 903 (e.g., braided wire mesh and/or mesh tube). In some examples, the device 902 may be configured for inflation of one or more gases and/or fluids delivered via the tube 910. The mesh 903 may be configured to facilitate longitudinal expansion of the balloon 901. For example, the mesh 903 may have a generally tubular form and/or may be configured to compress and/or expand longitudinally (e.g., along a direction of insertion and/or delivery of the device 902. The mesh 903 may comprise a wire composed of one or more metals (e.g., Nitinol and/or other shape memory alloys) and/or other suitable materials. The mesh 903 may form a tube and/or may comprise a number of interwoven wires forming a tubular shape. In some examples, the mesh 903 may be configured to be compressed to a compact form in which the wires compress closely together. The mesh 903 may be configured to expand and/or to be expanded to the expanded form shown in FIG. 9, in which the wires are generally spaced apart. As the mesh 903 expands, the mesh 903 may exert longitudinal force on the balloon 901 to cause the balloon 901 to form a generally oval shape, in which the balloon 901 has a greater length (e.g., longitudinal axis) than width (e.g., lateral axis). Thus, the mesh 903 may be configured to direct expansion of the balloon 901 longitudinally.

The device 902 can comprise an inflatable and/or expandable balloon 901. In some examples, the balloon 901 may be configured for inflation of one or more gases and/or fluids delivered via the tube 910. The balloon 901 may be configured to inflate to a generally spherical shape. However, the balloon 901 can have a generally flexible and/or pliable form and/or may be configured to conform to various environments and/or anatomies. The balloon 901 can be composed of various suitable materials, which can include compliant and/or non-compliant materials. Various materials of the balloon 901 may be configured to stretch and/or expand in response to filling of gases and/or fluids. In some examples, the balloon 901 may have sufficient elasticity to apply compressive force to the mesh 903. However, as the balloon 901 is inflated, the mesh 903 may be allowed to expand and/or shape the balloon 901 into an elongate oval shape.

The mesh 903 may be disposed at least partially and/or fully within the balloon 901. Accordingly, expansion and/or compression of the mesh 903 may be configured to cause corresponding expansion and/or compression of the balloon 901.

In some examples, the device 902 and/or tube 910 can comprise one or more additional features, which can include one or more pressure sensors configured to sense changes in preload and/or afterload, radiopaque markers, electrical leads for ventricular pacing, defibrillation, and/or permanent and/or temporary tissue anchors configured to facilitate placement and/or positioning of the device 902.

The device 902 may be configured to be selectively expanded and/or inflated. For example, the device 902 may be configured to inflated in synchronization with a cardiac rhythm and/or cycle. For example, the one or more sensors may be configured to generate and/or transmit data relating to cardiac rhythm (e.g., systole and/or diastole) and/or the device 902 may be automatically expanded and/or compressed on a periodic basis in synchronization with the cardiac rhythm.

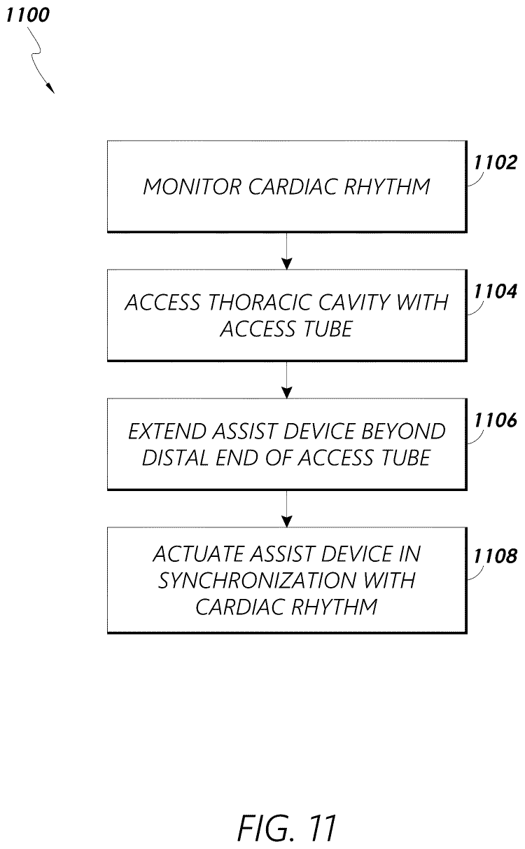

FIG. 10 illustrates an example assist device 1002 and/or actuator configured for delivery via an access tube 1010 (e.g., catheter and/or delivery shaft) in accordance with one or more examples. The device 1002 can comprise a mechanical actuation mechanism, which can include a scissor jack 1004 and/or linear pistons configured to cause extension and/or retraction of a distal hand 1006.

The device 1002 may be configured to direct the distal hand 1006 towards the heart 1 and/or right ventricle 4. In some examples, the device 1002 may be longitudinally expandable and/or may not be laterally expandable.

The hand 1006 may have any suitable shape and/or size. In some examples, the hand 1006 may have a generally concave, convex, and/or curved form. The hand 1006 may have a curvature configured to approximate a curvature of the heart 1 at or near the right ventricle 4. For example, the hand 1006 may be configured to extend along a curved portion of the heart. Accordingly, the hand 1006 may be convex relative to the tube 1010 and/or scissor jack 1004. The hand 1006 may form a cradle and/or may be configured to apply atraumatic pressure to a heart and/or other tissue. The scissor jack 1004 may operate passively, mechanically, and/or pneumatically.

The scissor jack 1004 may comprise an extender comprising interlocking arms 1008. The arms 1008 may be joined together by one or more joints 1009. In some examples, the jack 1004 may comprise a first set 1012 of arms 1008 and/or a second set 1014 of arms 1008. The first set 1012 of arms 1008 and/or the second set 1014 of arms 1008 may comprise multiple arms 1008 arranged in a zig-zag form. For example, the various arms 1008 of the first set 1012 and/or second set 1014 may be joined at ends of the arms 1008 by the joints 1009. The first set 1012 and the second set 1014 may extend in opposite directions and/or at an approximately 135-degree angle relative to each other. The arms 1008 of the first set 1012 may cross over arms 1008 of the second set 1014. In some examples, arms 1008 of the first set 1012 may be attached to arms 1008 of the second set 1014 via joints 1009 at intersection points 1016 between the first set 1012 and the second set 1014.

In some examples, the device 1002 and/or tube 1010 can comprise one or more additional features, which can include one or more pressure sensors configured to sense changes in preload and/or afterload, radiopaque markers, electrical leads for ventricular pacing, defibrillation, and/or permanent and/or temporary tissue anchors configured to facilitate placement and/or positioning of the device 1002.

The device 1002 may be configured to be selectively expanded and/or inflated. For example, the device 1002 may be configured to inflated in synchronization with a cardiac rhythm and/or cycle. For example, the one or more sensors may be configured to generate and/or transmit data relating to cardiac rhythm (e.g., systole and/or diastole) and/or the device 1002 may be automatically expanded and/or compressed on a periodic basis in synchronization with the cardiac rhythm.

Movement of the various cardiac assist devices described herein may be facilitated using one or more electromagnets and/or magnetic elements. For example, a cardiac assist device may comprise one or more magnetic elements and/or may be configured to assume a compressed form by default. An opposing magnetic clement may be brought into a magnetic field of the one or more magnetic elements of the cardiac assist device to cause expansion and/or inflation of various components (e.g., coils, scissor jacks, wires, wire forms, etc.) of the cardiac assist devices. The opposing magnetic element may be disposed outside the patient's body and/or within the patient's body but detached and/or distally connected to/from the cardiac assist device.

FIG. 11 provides a flowchart illustrating an example process 1100 for assisting cardiac performance in accordance with one or more examples. At block 1102, the process 1100 involves monitoring cardiac rhythm. Cardiac rhythm can include any physical measurement associated with cardiac function, including heartbeat rate, ventilation and/or breathing rate, and/or hemodynamic pressures at the thoracic cavity. Such measurements may be collected using various sensors within and/or outside the patient's body. For example, finger cuffs, pulse-sensing wristbands, and/or other devices may be used. The various sensors may be configured to transmit and/or convey measurement data to a control module configured to receive and/or store the measurement data.

At block 1104, the process 1100 involves accessing the patient's thoracic cavity using one or more access tubes and/or similar devices. The thoracic cavity may be accessed at any suitable location, which can include the anterior mediastinum. In some examples, a point of access may be at or near the right ventricle of the heart to assist in compression and/or expansion of the right ventricle. Any number of access tubes may be used and/or the thoracic cavity may be accessed at multiple points. The one or more access tubes may be coupled to a control module configured to establish an air-tight seal with the one or more access tubes. Moreover, the one or more access tubes may be configured to form an air-tight and/or fluid-tight seal at the patient's skin. In some examples, one or more sensor devices may be attached to the one or more access tubes.

At block 1106, the process 1100 involves extending an assist device and/or implant at least partially beyond a distal end of the access tube. The assist device may be at least partially expandable. At removal from the access tube, the assist device may be at least partially compressed and/or in an at least partially compressed form. For example, the implant may comprise an at least partially deflated balloon and/or ridged device in the compressed form. In another example, the implant may comprise an at least partially compressed scissor jack mechanism in the compressed form.

At decision block 1108, the process 1100 involves actuating the assist device. In some examples, actuating the assist device may involve inflating and/or extending the assist device. The assist device may be actuated in synchronization with a sensed cardiac rhythm.

### Additional Examples

Provided below is a list of examples, each of which may include aspects of any of the other examples disclosed herein. Furthermore, aspects of any example described above may be implemented in any of the numbered examples provided below.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Example 1: A method of assisting cardiac performance comprising accessing a thoracic cavity near a heart with an access tube, delivering, via the access tube, an expandable implant in a compressed form into the thoracic cavity, and expanding the expandable implant to cause compression of the heart.

Example 2: The method of any example herein, in particular example 1, further comprising monitoring cardiac rhythm using one or more sensors attached to the access tube or expandable implant.

Example 3: The method of any example herein, in particular example 2, further comprising expanding the expandable implant in synchronization with the cardiac rhythm.

Example 4: The method of any example herein, in particular example 2, wherein at least one pressure sensor is situated at an exterior surface of the access tube.

Example 5: The method of any example herein, in particular example 1, further comprising accessing the thoracic cavity via an anterior mediastinum with the access tube.

Example 6: The method of any example herein, in particular example 1, further comprising positioning a distal end of the access tube at or near a right ventricle of the heart within the thoracic cavity.

Example 7: The method of any example herein, in particular example 1, wherein expanding the expandable implant comprises delivering a fluid or gas into the expandable implant via the access tube.

Example 8: The method of any example herein, in particular example 7, wherein the expandable implant comprises a balloon.

Example 9: The method of any example herein, in particular example 8, wherein the expandable implant comprises a coil disposed inside the balloon.

Example 10: The method of any example herein, in particular example 8, wherein the expandable implant comprises a mesh tube disposed inside the balloon.

Example 11: The method of any example herein. in particular example 7, wherein the expandable implant comprises a ridged device configured to expand longitudinally and not laterally.

Example 12: The method of any example herein, in particular example 1, wherein the expandable implant comprises a scissor jack mechanism.

Example 13: A system comprising an expandable implant configured to move between an expanded form and a compressed form, and contact, at least in the expanded form, a heart via a thoracic cavity to cause compression of the heart.

Example 14: The system of any example herein, in particular example 13, wherein the expandable implant further comprises one or more sensors attached to the expandable implant, the one or more sensors configured to monitor cardiac rhythm.

Example 15: The system of any example herein, in particular example 13, further comprising an access tube configured to extend through the thoracic cavity to deliver the expandable implant.

Example 16: The system of any example herein, in particular example 13, wherein the expandable implant comprises a balloon.

Example 17: The system of any example herein, in particular example 16, wherein the expandable implant comprises a coil disposed inside the balloon.

Example 18: The system of any example herein, in particular example 16, wherein the expandable implant comprises a mesh tube disposed inside the balloon.

Example 19: The system of any example herein, in particular example 13, wherein the expandable implant comprises a ridged device configured to expand longitudinally and not laterally.

Example 20: The system of any example herein, in particular example 13, wherein the expandable implant comprises a scissor jack mechanism.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for case of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another clement having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A system for assisting cardiac performance, the system comprising:
a delivery tube;
an expandable implant sized for delivery within the delivery tube, the expandable implant being movable between an expanded form and a compressed form in a periodic manner to extend beyond the delivery tube and contact, at least in the expanded form, a heart via a thoracic cavity to cause compression of the heart; and
a sensor to measure cardiac rhythm, wherein the expandable implant moves in synchronization with the measured cardiac rhythm.

2. The system of claim 1, wherein the sensor is coupled to the expandable implant.

3. The system of claim 1, wherein the sensor is disposed along a surface of the delivery tube.

4. The system of claim 1, wherein the delivery tube is sized to extend through the thoracic cavity to deliver the expandable implant.

5. The system of claim 1, wherein the expandable implant comprises a balloon.

6. The system of claim 5, wherein the expandable implant comprises a coil disposed inside the balloon to facilitate expansion of the balloon.

7. The system of claim 5, wherein the expandable implant comprises a mesh tube disposed inside the balloon to support the balloon.

8. The system of claim 1, wherein the expandable implant comprises a ridged device that expands longitudinally and not laterally.

9. The system of claim 1, wherein the expandable implant comprises a scissor jack mechanism.

10. A system for assisting cardiac performance, the system comprising:
an expandable implant comprising a balloon and a support device disposed within the balloon, the expandable implant being movable between an expanded form and a compressed form in a periodic manner to contact, at least in the expanded form, a heart via a thoracic cavity to cause compression of the heart; and
a sensor to measure cardiac rhythm, wherein the expandable implant moves in synchronization with the measured cardiac rhythm.

11. The system of claim 10, wherein the sensor is coupled to the expandable implant.

12. The system of claim 10, wherein the support device comprises a coil disposed inside the balloon to facilitate expansion of the balloon.

13. The system of claim 10, wherein the support device comprises a mesh tube disposed inside the balloon to facilitate expansion of the balloon.

14. A system for assisting cardiac performance, the system comprising:
a sensor to measure cardiac rhythm; and
an expandable implant being movable between an expanded form and a compressed form in a periodic manner and in synchronization with the measured cardiac rhythm to contact, at least in the expanded form, a heart via a thoracic cavity to cause compression of the heart, wherein the expandable implant is structured to expand further longitudinally than laterally.

15. The system of claim 14, further comprising a delivery tube sized to deliver the expandable implant and the sensor.

16. The system of claim 14, wherein the expandable implant comprises a balloon.

17. The system of claim 16, wherein the expandable implant comprises a coil disposed inside the balloon to facilitate expansion of the balloon.

18. The system of claim 16, wherein the expandable implant comprises a mesh tube disposed inside the balloon to support the balloon.

19. The system of claim 14, wherein the expandable implant comprises a ridged device that expands longitudinally and not laterally.

20. The system of claim 14, wherein the expandable implant comprises a scissor jack mechanism.
